(19) United States
(12) Patent Application Publication
(10) Pub. No.: US 2020/0347379 A1
(43) Pub. Date: Nov. 5, 2020

(54) METHOD FOR ISOLATION OF NUCLEIC ACIDS

(71) Applicant: Phoenix Molecular Pte Ltd, Ayer Rajah Industrial Estate (SG)

(72) Inventor: Chung-Pei Ou, Aspen Heights #03-05 (SG)

(21) Appl. No.: 16/764,282

(22) PCT Filed: Nov. 13, 2018
(86) PCT No.: PCT/SG2018/050566

§ 371 Date: May 14, 2020

(30) Foreign Application Priority Data
Nov. 14, 2017 (GB) 1718803.8

(51) Int. Cl. C12N 15/10; C12Q 1/6806

(52) U.S. Cl. CPC C12N 15/1006; C12Q 2527/125; C12Q 1/6806

(57) ABSTRACT

The current invention provides methods and kits to isolate nucleic acids, including for purification of nucleic acids and nucleic acid enrichment, as well as for identification of nucleic acids in samples where they serve as a biomarker. More specifically, the invention provides a step of binding nucleic acid to a nucleic acid binding molecule which is either (a) a cationic molecule which is an amine, amidine or other amine-derived cation, or (b) a minor groove nucleic acid-binding molecule bound to a solid support and a step of dissociating bound nucleic acids from the nucleic acid-binding molecule by contact with a polyoxometalate or an oxalate.

### FIELD OF INVENTION

The current invention relates to isolation of nucleic acids including for purification of nucleic acids and nucleic acid enrichment, as well as for identification of nucleic acids in samples where they serve as a biomarker.

### BACKGROUND OF THE INVENTION

The listing or discussion of an apparently prior-published document in this specification should not necessarily be taken as an acknowledgement that the document is part of the state of the art or is common general knowledge.

The most common method of isolating nucleic acids is the silica method, which is derived from Boom's method (U.S. Pat. No. 5,234,809). The method is based on a unique reversible interaction between silica and nucleic acids. There are a number of variations, which relate to the design of the surface structure of the silica or how the silica is coupled to a solid matrix. For example, the hydration state of the silica can be changed to improve nucleic acid binding. Silica has been coupled to various substrates to form silica membranes and silica coated magnetic particles.

Boom's method is popular because the procedure is simple and the purity of nucleic acids isolated by the process is acceptable for most downstream applications. Boom's method comprises three steps: binding, washing and elution. However, where biological samples are concerned sample homogenization is usually undertaken before the first step. Nucleic acids are typically protected within a cell membrane and/or a cell wall, or a viral capsule. The nucleic acids will also generally be associated with proteins, e.g. histone protein or enzymes including enzymes that can digest nucleic acids. Therefore, in order to extract nucleic acid from a biological sample, cells must be lysed and enzymes that can modify or digest nucleic acid may need to be inactivated selectively. Further, when ribonucleotide acid (RNA) is not desired in the product, an enzyme for digesting RNA is usually included in the lysis step. The enzyme will hydrolyse RNA into individual nucleotides, which will be removed during the washing step and leave only deoxyribonucleic acid (DNA) in the eluted product. Equally, if RNA is the desired product, DNA can be selectively digested by including enzymes that digest DNA. If RNA is the desired product an RNAase inhibitor is often used to protect the RNA from degradation

A chaotropic agent is frequently added to break down the tertiary structure and ensure the release of nucleic acid from in the cellular components or viral capsules into the solution. Typically a chaotropic salt, e.g. guanidinium chloride, is added. The chaotropic salt also acts as an inhibitor to enzymes that will modify or hydrolyse nucleic acids, especially DNA. The salt concentration is crucial for nucleic acid binding to the silica surface. Silica does not capture nucleic acid efficiently when the salt concentration is low, and so salts such as guanidinium salt, sodium chloride, sodium acetate and potassium chloride are added to increase the ionic strength and so facilitate capture. The concentration of the salt in the sample solution after lysis and before the binding step is from 0.5M to 2M in Boom's method. Nucleic acids are captured on the silica surface in the binding step, while proteins, lipids, and other cellular components are not, or bind only weakly through non-specific interactions. However salt is also captured on the silica matrix. The silica matrix is then washed with high concentration of alcohol (usually about 70% ethanol) in the washing step to remove any weakly bound cellular components and/or any salt that has been retained. However, complete removal of the alcohol from the nucleic acids is difficult.

It is well known that alcohol is a strong inhibitor to many enzymes in downstream applications, for example polymerases used in PCR, the ligases used in annealing reactions, and nucleases used in restriction enzyme digestion. Therefore additional steps are required to ensure removal of the alcohol from the matrix so that it does not interfere with downstream applications. The methods used include heating the silica matrix, centrifuging the matrix at high speed, or exposing the matrix to air to allow the alcohol to evaporate. After removing the residual alcohol, the nucleic acid is eluted from the silica surface with a solution that has a low salt concentration, e.g. 10 mM Tris and 1 mM EDTA, pH8.0.

Solvent-free methods for nucleic acid isolation employing ion-exchange chromatography have been developed. Anion exchange chromatography has been used to purify nucleic acids for a long time. The method uses a cationic resin which binds to nucleic acids, which are negatively charged. Nucleic acids such as genomic DNA or plasmids have tens of thousands of negatively charged phosphate groups along their length. Therefore a cationic resin binds very strongly to a nucleic acid. On the other hand, protein, lipid, and oligosaccharides are either zwitterionic, positively charged or carry far less negative charge, and so do not bind to the cationic resin as strongly as nucleic acids. The affinity difference between nucleic acids and other cellular components is exploited to extract nucleic acids. In the process an ionic solution containing a counter-ion, e.g. sodium chloride, guanidinium chloride, guanidine isothiocyanate, or potassium chloride, is passed through the resin and the concentration of the counter-ion is increased gradually. Molecules that bind most weakly will be eluted at the lowest concentration. As the concentration of counter-ion increases, more tightly bound molecules are eluted. Typically, proteins, oligosaccharides and lipids are eluted below 0.3M sodium chloride. Nucleic acids may be eluted from 0.5M to 2M of sodium chloride or higher. However when nucleic acids are eluted with salt solutions, large amounts of salt also moves with nucleic acid into the eluate. The concentration of the salt is so high that it will inhibit most downstream applications. Therefore additional steps are required to remove the salt from the nucleic acid solution.

Thus, there is a need for a process for isolating nucleic acids which does not require the removal of contaminants such as alcohol and salts before downstream processing is undertaken. The present disclosure aims at providing such a process.

### SUMMARY OF THE INVENTION

According to a first aspect of the invention there is provided a process for the separation of a nucleic acid from a first nucleic acid-containing composition, comprising the steps of:
a) providing the first nucleic acid-containing composition in a first phase;
b) providing a nucleic acid binding molecule which is either (a) a cationic molecule which is an amine, amidine or other amine-derived cation, or (b) a minor groove nucleic acid-binding molecule in a second phase;
c) allowing contact to be maintained between the first phase and the second phase for a sufficient time for nucleic acids in the first nucleic acid-containing composition to bind to the nucleic acid-binding molecule;
d) separating the first phase from the second phase; and
e) treating the second phase to dissociate bound nucleic acids from the nucleic acid-binding molecule by contact with a dissociation composition comprising a polyoxometalate or an oxalate molecule to produce a second nucleic acid-containing composition.

According to a second aspect of the invention there is provided a process according to the first aspect further comprising amplifying one or more of the nucleic acids present in the second nucleic acid-containing composition.

According to a third aspect of the invention there is provided a process according to the first aspect further comprising amplifying one or more of the nucleic acids present in the second nucleic acid-containing composition, and detecting the presence of a specific nucleic acid or of nucleic acids generally.

According to a fourth aspect of the invention there is provided a device for the separation of a nucleic acid from a first nucleic acid-containing composition configured for use in the process of any of the first to third aspects.

According to a fifth aspect of the invention there is provided a kit comprising a first composition comprising a nucleic acid binding molecule which is either (a) a cationic molecule which is an amine, amidine or other amine-derived cation, or (b) a minor groove nucleic acid-binding molecule; and a second composition comprising a polyoxometalate or an oxalate, wherein addition of the second composition to nucleic acids bound to the nucleic acid binding molecule results in disassociation of the nucleic acids from the nucleic acid binding molecule.

### BRIEF DESCRIPTION OF THE DRAWINGS

In order that the disclosure may be readily understood and put into practical effect, reference will now be made to example embodiments as illustrated with reference to the accompanying figures. The figures together with the description serve to further illustrate the embodiments of the invention and explain various principles and advantages.

FIG. 1 is a graph showing speciation of vanadate (V) against pH. The distribution of species depends on both the concentration of vanadium and the pH of the solution. At higher concentrations, the majority of the vanadium is in the form of a decamer (HV₁₀O₂₈)₅ at a pH of 5.

FIG. 2 shows images of results of DNA separation using Streptomycin-resin. (a) Photograph of DNA electrophoresis gel with molecular weight scale for pBR322 Msp I digested DNA (b) Photograph of a DNA electrophoresis gel where pBR322 Msp I digested DNA has been isolated using a streptomycin-conjugated resin. The gel has a lane for a positive control (M), flow through fraction (FT0), wash fraction (W0) and recovered nucleic acid fraction (E0).

FIG. 3 shows a photograph of a DNA electrophoresis gel where pBR322 Msp I digested DNA has been isolated using a DEAE resin. The gel has a lane for a positive control (M5), flow through fraction (FT2), wash fraction (W2) and recovered nucleic acid fraction (E2).).

FIG. 4 shows a graph of CT value against log (copy number) where 2000 copies of mouse genomic DNA is eluted from a streptomycin-conjugated resin.

FIG. 5 shows a graph comparing various nucleic acid samples to the sodium chloride (NaCl) concentration at which NaCl elutes DNA from a streptomycin-conjugated resin.

FIG. 6 shows a photograph of a DNA electrophoresis gel where pBR322 Msp I digested DNA has been eluted with sodium vanadate (Lane 1), potassium oxalate (Lane 2), tartrate tetra-hydrate (Lane 3), hexachloroplatinate (IV) (Lane 4), and sodium hydroxide (Lane 5).

FIG. 7 shows a photograph of a DNA electrophoresis gel where pBR322 Msp I digested DNA has been eluted with decavanadate (Lane 1E), sodium tungstate (Lane 2E) or various forms of sodium molybdate (MoO4(2-) pH 6.1, Lane 3E; Mo7O24(6-) pH 4.1, Lane 4E and Mo8O26(4-) pH 2.1, Lane 5E). (F) represents collected flow through; (W) represents collected 0.5 M NaCl wash prior to elution; (E) represents collected decavanadate, sodium tungstate or sodium molybdate eluate; (S) represents collected 2.5 M NaCl wash after the elution of DNA.

### DEFINITIONS

For convenience, certain terms employed in the specification, examples and appended claims are collected here.

The term “first phase”, as used in the context of the invention refers to a liquid or mobile phase which comprises a nucleic acid-containing composition.

The term “comprising” is herein defined to be that where the various components, ingredients, or steps, can be conjointly employed in practicing the present invention. Accordingly, the term “comprising” encompasses the more restrictive terms “consisting essentially of” and “consisting of.”

### DETAILED DESCRIPTION OF THE INVENTION

The current invention relates to a process for the isolation of a nucleic acid from a first nucleic acid-containing composition. The first nucleic acid-containing composition is brought into contact with a nucleic acid binding molecule which is a cationic molecule which is an amine, amidine or other amine-derived cation, or a minor groove nucleic acid-binding molecule. Contact is maintained for a sufficient time for nucleic acids in the first nucleic acid-containing composition to bind to the nucleic acid-binding molecule. Other components of the composition either do not bind or are washed away. Thereafter bound nucleic acids are dissociated from the nucleic acid-binding molecule by contact with a dissociation composition comprising a polyoxometalate, oxalate or a soluble minor groove binding molecule to produce a second nucleic acid-containing composition. In general the first nucleic acid-containing composition is derived from a biological sample and so contains other biological molecules such as proteins and lipids, and the second nucleic acid-containing composition is substantially free of these components.

The process is compatible with downstream enzymatic reactions. There is no need to remove alcohol or salt, as in other elution methods. This means that amplification of the nucleic acids is simplified as the eluted DNA/RNA can be used directly in an amplification step. This avoids the need for undertaking further processing steps which are time-consuming and potentially allow for contamination of a sample. Therefore the detection of nucleic acids generally or of a specific nucleic acid in a sample from a patient is simplified.

The first nucleic acid-containing composition can be a biological sample, e.g. fluid, solid, cultured cell, stool, hair, swab, or a sample taken from an environment such as a forensic sample taken from clothes, a wall swab, plastic, fabric, soil or the like.

The biological sample may contain intact cells, which are lysed prior to nucleic acid extraction. The lysis step could be mechanical, thermal, enzymatic, or chemical methods, or a combination of these, as would be well understood by the person skilled in the art. Suitable mechanical methods include sonication, disruption by inert abrasive particles, or lysis by sheer force. Thermal methods include direct heating or freeze and thaw cycle. Chemical methods include lysis by the addition of chaotropic salt, solvent, or osmotic shock-inducing salts. Enzymatic methods include hydrolysis with a proteinase.

According to a preferred embodiment a nucleic acid binding molecule is bound to a solid support, which could be a matrix, a membrane, or a surface. Typically the nucleic acid binding molecule is packed into a column to which the first nucleic acid-containing composition is introduced. The solid support forms a second phase, referred to as the stationary phase, while the first nucleic acid-containing composition forms a first phase, referred to as the mobile phase. The nucleic acid binding molecule is attached to the stationary phase with the nucleic acid binding portion available in solution. During the extraction, the nucleic acid binding molecule will capture the nucleic acid, and that becomes part of the stationary phase temporarily. As would be well understood by the person skilled in the art, such a column is designed to allow contact to be maintained for a sufficient time for nucleic acids in the first nucleic acid-containing composition to bind to the nucleic acid-binding molecule. Other components of the first nucleic acid-containing composition either pass through the column without binding, or are weakly bound and so may be washed from the column in a washing step while the nucleic acids remain bound.

Although it is not necessary and not preferred, the first nucleic acid-containing composition may contain a salt. As will be well understood by the person skilled in the art, addition of a salt interferes with intramolecular interactions mediated by non-covalent forces such as hydrogen bonds, van der Waals forces, and hydrophobic effects so as to reduce or eliminate non-specific binding to the nucleic acid binding molecule. The salt may be any salt which reduces or eliminates non-specific binding to the nucleic acid binding molecule. In an embodiment the salt is selected from the group consisting of sodium chloride, sodium acetate, potassium chloride, hexadecyltrimethylammonium bromide (CTAB), histidine, urea, guanidine hydrochloride, guanidine isothiocyanate, alkaline solution, and ammonium chloride. The salt may be added as an aqueous solution to inhibit non-specific binding to the nucleic acid binding molecule. At lower concentration, say, 0.1 M for example, but in a range from 0.1 to 0.5 M, it inhibits proteins, lipids or other non-nucleic acid molecules from associating with the nucleic acid binding molecule. At higher salt concentration, smaller nucleic acids are removed, such that only larger nucleic acids are selectively captured.

A salt solution with a concentration of from 0.1 to 1M may also be used to wash a column to remove non-specific bound molecules and then be discarded. There may be from 1 to 6 washing steps but usually only 1 or 2 washing steps are necessary. One or more different salts may be mixed in a solution. Washing may be effected with different salt solutions applied in sequence, if desired. By way of example, a guanidinium chloride solution may be employed in the first washing step followed by water in a second washing step to remove any residual guanidinium chloride. Where the first nucleic acid-containing composition contains a salt, or a salt solution is used for washing, there will generally be a final wash with water to remove any residual salt prior to the elution step.

In another preferred embodiment the cationic molecule is a cationic polymer.

In another preferred embodiment the cationic polymer is a polyethyleneimine, poly-lysine, poly-arginine, guanidino polymer, or an amidine polymer. A guanidino polymer may be, for example, a condensation polymer formed by polymerization of a diamine, such as hexamethylene diamine, and guanidine hydrochloride with subsequent crosslinking, for example, using epichlorohydrin.

In another preferred embodiment the cationic polymer is a polyethyleneimine.

In another preferred embodiment the cationic molecule is immobilised to a solid support.

In another preferred embodiment the cationic molecule is an amine. For example the amine may comprise a primary amine, or the diethylaminoethyl (DEAE), trimethylammonium (QMA) or diethyl-(2-hydroxypropyl)aminoethyl (QAE) groups, immobilised to a solid support.

The support may be any of solid support capable of packing a column, as would be well understood by the person skilled in the art. By way of example, the solid support may be Sephadex, cellulose, a silica oxide surface, a metal oxide surface, a ceramic, Sepharose, agarose, cross-linked agarose, core-shell particles, magnetic particles, dextran, cross-linked dextran, acrylamide, cross-linked acrylamide, or other polymer particles, e.g. polystyrene, polyethylene.

In another preferred embodiment the nucleic acid binding molecule is a minor groove nucleic acid-binding molecule.

In another preferred embodiment the nucleic acid binding molecule is a resin comprising a solid support to which a minor groove nucleic acid-binding molecule has been immobilised.

In another preferred embodiment the minor groove nucleic acid-binding molecule is molecule carrying one more groups selected from amidino, amido, or guanidino, such as a conjugate derived from 4-aminobutyl guanidine, arginine or 4-aminobutylguanidine, which binds the minor groove of DNA.

In another preferred embodiment the minor groove nucleic acid-binding molecule contains an amidino group.

In another preferred embodiment the minor groove nucleic acid-binding molecule is an antibiotic, an antimicrobial agent, or an anti-cancer agent.

In another preferred embodiment the minor groove nucleic acid-binding molecule is an antibiotic, or a derivative thereof.

In another preferred embodiment the antibiotic is streptomycin or a derivative thereof (see, for example, Liu, Y. J, Am Chem Soc., 133(26): 10171-10183 (2011), the contents of which are incorporated herein be reference).

In another preferred embodiment the antibiotic is netropsin, DAPI, tetracycline, Hoechst 33258, Neomycin, or an isoquinoline alkaloid such as palmatine, coralyne or berberine.

The amidine or amide group of many antibiotics is important in their ability to bind nucleic acids. The amidine could interact with nucleic acid through a stacking effect and H-bonding. [Liu, Y. J, Am Chem Soc., 133(26): 10171-10183 (2011)]. Derivatives of streptomycin were developed in this paper to study the binding of the nucleic acid. Other antibiotics are also know to bind to nucleic acids, such as Netropsin, DAPI, tetracycline, Hoechst 33258, Neomycin binding to poly A of RNA [Xi, H. FEBS Letters 583: 2269-2275 (2009)], isoquinoline alkaloids [Gin, P. Cytotoxic plant alkaloid palmatine binds strongly to poly(A), Bioorg. Med. Chem. Lett. 16: 2364-2368(2006); Xing, F. Coralyne binds strongly to poly(A), FEBS Lett. 579: 5035-5039 (2005); Yadav, R. C., Berberine, a strong polyriboadenylic acid binding plan alkaloid, Bioorg. Med. Chem. 13: 165-174 (2005)]. These structural studies show that the association occurs at the minor groove of nucleic acids.

Other nucleic acid binding molecules could also be use, such as other antibiotic drugs or anti-cancer drugs, these are known to bind to nucleic acid through stacking interactions, hydrogen bonding, and electrostatic attractions.

In another preferred embodiment minor groove nucleic acid-binding molecule is capable of targeting specific sequences. In an embodiment the minor groove nucleic acid-binding molecule is capable of selecting the A-T rich nucleic acids. In an embodiment the nucleic acid binding agent specifically capable of selecting the A-T rich nucleic acids is an amidine. Many amidine-based nucleic-acid binding agents bind to A-T rich sequence with much higher affinity than random or G-C rich sequence. For example, bis-amidinocarbazoles have a planar chromophore capable of reading the genetic information accessible within the minor groove of AATT sequence [Tanious, F. A., et al., Eur J Biochem. 268(12): 3455-64 (2001)]. Carbazole derivatives have been shown to bind to nucleic acids much more strongly than the corresponding dibenzothiophene and dibenzofurans. It has shown that the cationic moieties of either amidines or imidazolines appeared to have an effect on strong binding to DNA and RNA [Martine Demeunynck, Christian Bailly and W. David Wilson—2006—John Wiley & Sons, ISBN: 978-3-527-60566-8]. Another dicationic diamidine derivate has been shown to have unusually strong binding to the minor groove of DNA where it contains the sequence AATT. In another study, the amidine group of Netropsin-oxazolopyridocarbazole showed stronger affinity to poly AT sequence than poly GC sequence. [Molecular Basis of Specificity in Nucleic Acid-Drug Interactions: Proceedings of the Twenty-Third Jerusalem Symposium on Quantum Chemistry and Biochemistry Held in Jerusalem, Israel, May 14-17, (1990) Eds. B. Pullman and J. Jortner, Springer Science+Busines Media, B.V.; ISBN 978-94-010-5657-1]. In yet another publication, a hexacyclic amidine analogue of Hoechst 33258 showed strong selective affinity to a sequence of GGTAATTACC [Bostock-Smith, C. E. et al., Chem. Commun., 0: 121-122 (1997)]. Therefore, the minor-groove binding agent may be used to enrich/extract a selected class of molecules or even a specific sequence in preference to other nucleic acids in the first nucleic acid-containing composition. In an embodiment binding to G-C rich or random sequences can be achieved but adjusting parameters such as salt concentration in the sample.

In another preferred embodiment the nucleic acid binding molecule is a resin to which a sequence-specific binding agent is bound. Where a sequence-specific binding agent is used enrichment for a nucleic acid carrying a target sequence which will bind to the sequence-specific binding agent is possible. The target sequence could be a biomarker.

In another preferred embodiment, the nucleic acid binding molecule can be present in first liquid phase that is not soluble or miscible with a second liquid phase in which the nucleic acid sample is present. The two liquid phases can be aqueous/aqueous or organic solvent/aqueous. Examples include chloroform/water, oil/water and PEG solution/water systems. The nucleic acid binding molecule exists only in first liquid phase, not in the second phase. The nucleic acid binding molecule can be part of a matrix in the first liquid phase or a soluble component in the first liquid phase. Binding to the nucleic acid can happen at the boundary of the first liquid phase and second liquid phase. In one embodiment, one of the liquid phases is a micro droplet in the other liquid phase. Washing and elution could be achieved by replacing the second liquid phase with a washing solution and then with an elution solution.

Water can be used to rinse and wash the second phase after nucleic acid binding molecule occurs in order to remove residual salt and also to remove non-specifically bound molecules. No alcohol is required in the process. Therefore, there is no need to dry the solid surface or to remove alcohol from the surface.

Dissociation of bound nucleic acids from the nucleic acid-binding molecule takes place by contact with the dissociation composition. When used in a chromatographic process the dissociation composition is referred to as an elution buffer. In such processes the elution buffer may comprise a polyoxometalate ion, oxalate or soluble minor groove binding molecules, or mixtures any of these compounds.

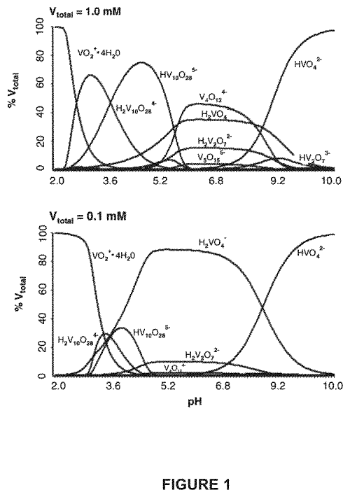

In another preferred embodiment the dissociation composition comprises a polyoxometalate. In aqueous solution these metal oxides undergo pH dependent condensation or dissociation and exist as a variety of chemical species. The phenomenon is called speciation. The composition of the species in solution depends on the pH of the solution and the concentration of the oxide. To prepare an oxide solution, one can dissolve a solid oxide in an alkaline solution. Under alkaline conditions, the oxide can exist as a single, discrete unit. As the pH decreases, the oxide in the solution forms polyatomic ions. Multiple species usually exist to give an equilibrium mixture, the precise nature of which depends on the pH and concentration of the oxide as shown in FIG. 1 [from Alan S. Tracey, Gail R. Willsky, and Esther S. Takeuchi, “Vanadium: chemistry, Biochemistry, Pharmacology and Practical Applications” by CRC Press, ISBN: 1-4200-4613-6, Page 21 (2007)]. While not wishing to be bound by theory, it is believed that the polyatomic anions form complexes with amine compounds; hence the polyoxometalates in the elution buffer elute nucleic acids by successfully binding to the nucleic acid binding molecule. For example, the polyatomic anions displace nucleic acids bound to amidine groups (such as in streptomycin) and to tertiary amine groups (such as in DEAE).

In another preferred embodiment the polyoxometalate is an oxide of a transition metal. Oxides of transition metals can form polymerised anions (polyoxometalate) in solution, such as tungsten, molybdenum, niobium, tantalum, and vanadium. In general the polyoxometalate will a mixture of species formed on dissolution of the polyoxometalate in alkaline solution followed by acidification. The nature of the species is pH-dependent. In an embodiment the final pH of the solution is from 4 to 8. In an embodiment the final pH of the solution is from 5 to 6. The nature of the species is also concentration-dependent. In an embodiment the concentration of the polyoxometalate in the solution is from 10 mM to 500 mM.

In another preferred embodiment the polyoxometalate is an oxide of tungsten, molybdenum, niobium, tantalum or vanadium.

In another preferred embodiment the polyoxometalate is a vanadate, typically NaVO₃.

In another preferred embodiment the dissociation composition comprises oxalate, typically (NH₄)₂C₂O₄.

In another preferred embodiment the dissociation composition comprises a soluble minor groove binding molecule. In an embodiment soluble minor groove binding molecules include bis-amidine carbazole dications, amidinocarbazoles, imidazole carbazoles, streptomycin, 2, 7-diamidinocarbazole, pentamidine, Netropsin, DAPI, Berenil, and Hoechst 33258, or mixtures thereof.

The second nucleic acid-containing composition does not require further purification, such as with a solvent or alcohol, before downstream application. A relatively low concentration of the polyoxometalate, oxalate or a soluble minor groove binding molecule is required to elute nucleic acids. In an embodiment, the concentration may be selected from the range of 0.01 to 500 mM. A typical concentration is from 10 to 500 mM. It is sometimes desired to recover nucleic acid in as small volume as possible. The volume of the eluate may be reduced by using a smaller volume of eluent having a higher concentration of polyoxometalate, oxalate or a soluble minor groove binding molecule.

The method has very high recovery yield. It is capable of capturing very dilute samples, for which the recovery is 50% to 90% (1000 copies in 1 mL blood). The method works within a wide size range and for various types of nucleic acid, both single stranded and double stranded. The process can be used to recover single stranded DNA which is as small as but not limited to 20 nucleotides or double stranded DNA which has as few as but not limited to 18 base pairs.

The invention also relates to a device for the isolation of a nucleic acid from a first nucleic acid-containing composition configured for use in the process of the invention. The device can take the form of a disposable integrated cartridge in order to minimize the cross-contamination between samples. The invention also provides for an integrated device for detection where the extraction step is a module within a device which also amplifies isolated nucleic acids and/or detects nucleic acids of interest.

Circulating nucleic acid is used as a biomarker for diagnostic and prognostic purposes as well as the management of treatment in many fields. These include cancer screening and treatment, fetal gene testing from maternal liquid, trisomy detection from maternal blood, and diabetes mellitus management. Nucleic acids such as DNA, mRNA, or micro RNA can be found in various body fluids, including saliva, cerebrospinal fluid, bronchial lavages, breast milk, tears, stool, urine, and semen. In other organisms, circulating nucleic acid can be found in animals, but also in plants (Gahan, P. B., 2003, Cell Biochem Funct 21: 207-209). In clinical application, it has been found that the DNA level in the blood increased in the event of trauma, sepsis, or treatment of tumor. In the case of oncology, circulating nucleic acid is being used for monitoring the condition of patients with an existing tumor as well as for the detection of cancer at earlier stage [Phallen, J. Science Translational Medicine 9: eaan2415 (2017)]. Detection of tumors at an early stage has been difficult due to the limited amount of nucleic acid shed from the tumor tissue. A large percentage of the circulating DNA fragments from cancer patients are smaller than 100 bps [Mouliere, F, PloS One 6(9): e23418 (2011)]. In the case of microRNA, their abundance in the blood sample is very low and they are very small in size (<25 nucleotides, single stranded). The available commercial products include miRNAeasy serum/plasma from Qiagen, miRCURY RNA isolation from Exiqon or mirVANA PARIS from Thermofisher. These products use ethanol or isopropanol to treat the sample and to wash the resin. Very little information is available regarding the recovery yield for very small nucleic acids.

The present invention has the ability to recover small-sized single stranded DNA which is 20 nucleotides or double stranded DNA which is 18 base pairs. The extraction and recovery is done without solvent or alcohol. This opens application for concentrating short nucleic acid fragments, single stranded or double stranded, from body fluid where the quantity of the nucleic acid fragment is very small.

The invention also relates to a kit to put into practice the process of the invention.

In a preferred embodiment such a kit comprises a first composition comprising a nucleic acid binding molecule which is either (a) a cationic molecule which is an amine, amidine or other amine-derived cation, or (b) a minor groove nucleic acid-binding molecule; and a second composition comprising a polyoxometalate or an oxalate. As described previously the nucleic acid binding molecule is generally immobilized on a solid support and will be present in the kit in this form. An alternative will be to provide the solid support and reagents for attachment of the nucleic acid-binding molecule to the solid support. The kit may further comprise buffers and reagents for isolating a nucleic acid and/or buffers and reagents for amplification and/or detection of nucleic acids. In addition, it may include a device for isolating and/or amplifying and/or detecting a nucleic acid.

In use, the first composition contained in the kit forms a second phase in a separation process for separating nucleic acids contained in a first phase from other components of the first phase. Generally the second phase is a stationary phase, and then the nucleic acid binding molecule is immobilized to a solid support. The second composition is used to dissociate the nucleic acid from the stationary phase, such as by eluting therefrom. Accordingly, it will be appreciated that addition of the second composition to nucleic acids bound to the nucleic acid binding molecule results in disassociation of the nucleic acids from the nucleic acid binding molecule.

Non-limiting examples which embody certain aspects of the invention will now be described.

### EXAMPLES

Standard molecular biology techniques known in the art and not specifically described were generally followed as described in Sambrook and Russel, Molecular Cloning: A Laboratory Manual, Cold Springs Harbor Laboratory, New York (2001).

### Example 1

### Preparation of Vanadate Solution

25 mmoles of NaVO₃ was dissolved in 25 mL of 1 Molar NaOH. The pH was slowly adjusted to between pH 5 and pH 6 and allowed to stand. After standing, the solution was diluted it to approximately 500 mM with water and then to 25 mM.

### Example 2

### Binding of Nucleic Acid Minor Groove Molecule-Conjugated Surface: Streptomycin and Elution by Vanadate Solution

Reagents: Glycolink kit from ThermoFisher, Streptomycin powder, Corning X-spin filter column, nucleic acids of various sizes (0.02 microg/microlitre pBR322 Msp I digested DNA from New England Biolab), 25 mM sodium vanadate solution pH 6.

Synthesis of streptomycin conjugated surface and elution was undertaken as follows:
1. 13 mg of streptomycin was conjugated to 300 μl of Glycolink resin (50% w/v) using the method set forth in the user manual for the resin.
2. A 20 μl aliquot of streptomycin-conjugated resin was packed into a filter column. The column was spun at 10 K rpm for 1 minute to remove storage liquid.
3. 20 μl of the pBR322 Msp I digested DNA was added to the resin in the column and spun at 10 K rpm for 1 minute to collect the flow through (FT0).
4. The column was washed with 25 μl water and spun to collect the wash (W0)
5. Nucleic acid was eluted with 20 μl of vanadate solution (E0)
6. A DNA electrophoresis was run on the flow through with a positive control (M) which has the same amount of pBR322 Msp I digested DNA as used in step 3.

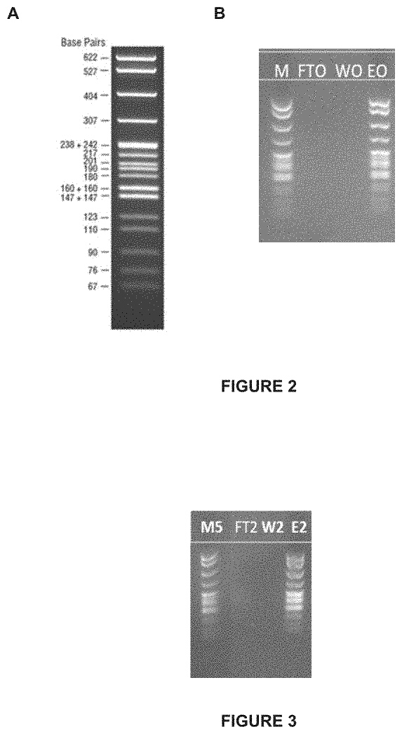

As shown in the photo presented in FIG. 2, nucleic acid is absent in flow through (FT0) and the wash (W0). The nucleic acid recovered from the column (E0) is close to the input (M) in terms of the DNA molecules contained therein. All the small fragments (67, 76, 90, 110, 123 bps) are recovered.

### Example 3

### Nucleic Acid Purification Using Cationic Exchange Surface and Vanadate: DEAE Resin

Reagents: DEAE resin from Qiagen Genomic-tip, Corning X-spin filter column, nucleic acids of various sizes (0.02 microg/microlitre pBR322 Msp I digested DNA from New England Biolab) 25 mM sodium vanadate solution pH6.

Preparation of DEAE column and elution was undertaken as follows:
1. 25 μl of DEAE resin was packed into a spin column and washed with QBT buffer from Qiagen kit.
2. 0.5 μg of pBR322 Msp I digested DNA was added to the DEAE resin according to the manual.
3. The sample was spun and the flow through (FT2) was collected.
4. The column was washed with 25 μl of QC buffer and the wash (W2) was collected.
5. Nucleic acids were eluted with 25 μl of vanadate solution and collected (E2).
6. DNA electrophoresis was run for the fractions collected together with a positive control (M5).

As shown in the photo reproduced in FIG. 3, nucleic acids are absent from the flow through (FT2) or wash (W2). The nucleic acid is eluted by vanadate without using a high concentration of salt.

### Example 4

qPCR Result for Vanadate Eluted Nucleic Acid from Streptomycin Surface

Reagents: streptomycin-conjugated resin, Corning X-spin filter column, mouse genomic DNA, qPCR 2× mastermix from Bioline, hydrolysis probe assay mix (Mm.PT.58.7161123.g) from IDT DNA service. 25 mM sodium vanadate solution pH 6. Human serum from Sigma Aldrich.

A streptomycin-conjugated column was prepared and used as follows:
1. 25 μl of streptomycin-conjugated resin was packed into a spin column.
2. 2000 copies of mouse genomic DNA were added to the spin column and spun.
3. The column was washed with 500 μl of water twice.
4. Nucleic acids were eluted with 25 μl of vanadate solution.
5. 1 μl of eluted DNA was mixed with 0.5 μl of assay mix, 5 μl of mastermix, and 3.5 μl of water. A qPCR assay was run on an ABI 7500Fast.

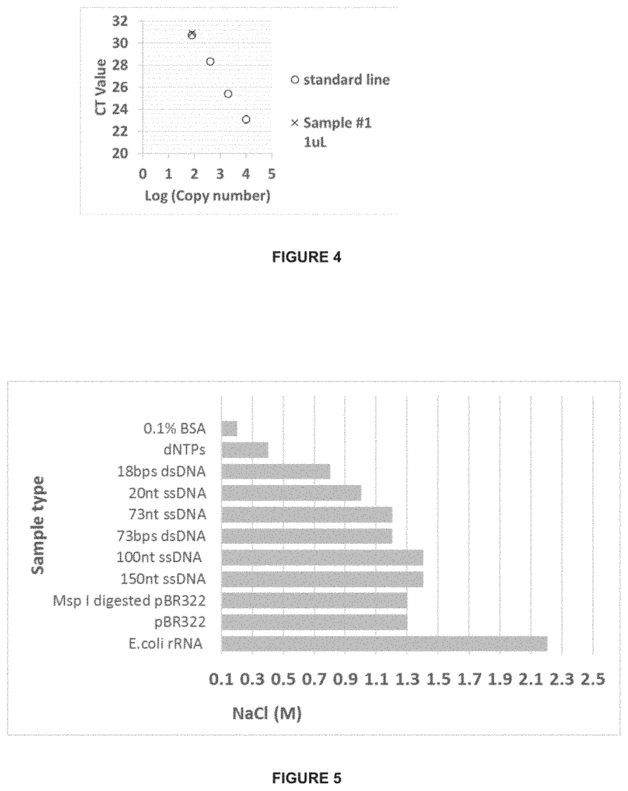

As FIG. 4 shows the eluant is compatible with downstream qPCR reaction. The recovery yield is close to complete.

### Example 5

### Streptomycin-Conjugated Resin Binds Nucleic Acids Tightly

Target reagents: 0.1% bovine serum albumin, dNTP (equal portion of dATP, dTTP, dGTP, and dCTP), 18 base paired double-stranded DNA, 20 nucleotide DNA oligo, 73 base-paired double-stranded DNA, 73 nucleotide DNA oligo, 100 nucleotides DNA oligo, 150 nucleotides DNA oligo, Msp I digested pBR322 plasmid, E. Coli ribosomal RNA.

Resin and elution reagents: streptomycin-conjugated resin as prepared in Example 1, and sodium chloride solution.
1. 0.4 μg of each of the target reagents was added separately to 25 μl of resin.
2. The sample was spun and the flow through fraction collected.
3. The resin was washed with 25 μl water and the wash fraction collected.
4. Elution with a sodium chloride gradient from 0.2 M to 2.4 M was performed.
5. The fraction from each gradient was collected.
6. All the fractions were run on agarose gel using gel electrophoresis.

As the graph of FIG. 5 shows, the minor-groove binding molecule conjugated resin (streptomycin) has specific and tight binding to nucleic acid, DNA and RNA. Smaller nucleic acid could be eluted by high salt at 0.8 M. Larger nucleic acid requires up to 2.2 M to be eluted from the resin.

### Example 6

### Elution of Nucleic Acid by Oxalate

Reagents: streptomycin conjugated resin (25% w/v) prepared as for Example 2, pBR322 plasmid DNA, sodium vanadate, potassium oxalate, 100 bp DNA ladder (NEB Biolab). Steps:
1. Elution solution 1 (50 mM sodium vanadate), elution solution 2 (50 mM potassium oxalate), Elution solution 3 (tartrate tetra-hydrate), Elution solution 4 (hexachloroplatinate (IV)) and elution solution 5 (1M NaOH) were prepared by dissolving the named reagent in water.
2. 5 tubes were prepared, one for elution with each of the elution solutions. 5 microlitres of streptomycin conjugated resin and 5 μl of 0.1 μg/μl of pBR322 plasmid DNA (undigested) were added to each tube.
3. The components were mixed well.
4. After mixing, elution solution 1 to 5 were added to tubes 1 to 5 respectively.
5. The resin mixture was transferred to an x-spin column and spun for 1 minute at 10 K rpm.
6. The eluted solution was examined by DNA gel electrophoresis. The DNA ladder marker is a 100 bps ladder.

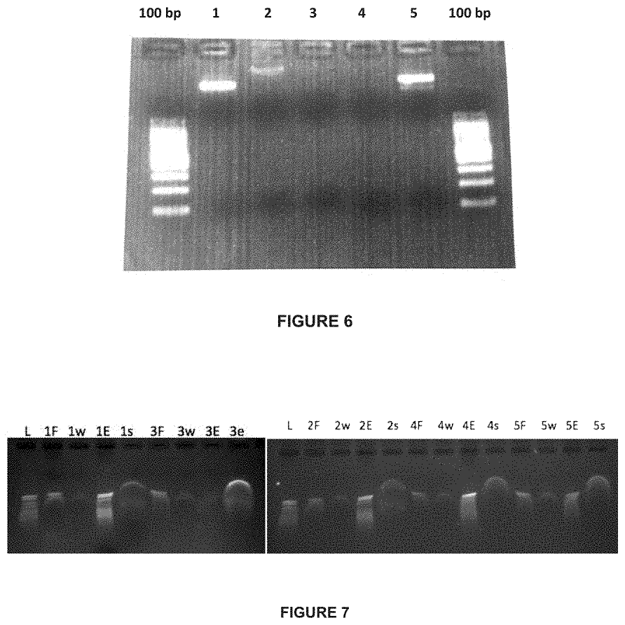

The gel photo (FIG. 6) showed that pBR322 could be eluted by sodium vanadate and potassium oxalate, as well as by using sodium hydroxide to increase pH and cause deprotonation.

### Example 7

### Elution of Nucleic Acid by Tungstate and Molybdate

Reagents: Glycolink kit from ThermoFisher, Streptomycin powder, Corning X-spin filter column, nucleic acids of various sizes (0.15 μg pBR322 Msp I digested DNA from New England Biolab), 50 mM sodium tungstate, 50 mM sodium molybdate.

### Steps:

1. Prepared streptomycin conjugated resin as described in Example 2.
2. Aliquoted 15 μl of streptomycin-conjugated resin to filter column. Spun to remove storage liquid.
3. Excess nucleic acid was added to achieve saturated nucleic acid binding sites. Added 15 μl of the pBR322 Msp I digested DNA in 0.5 M sodium chloride to the resin in the column and centrifuged to collect the flow through (F). 0.5 M sodium chloride prevent non-specific interaction of nucleic acid.
4. Washed the column with 15 μl of 0.5 M NaCl and spin to collect the wash (W)
5. Elute nucleic acid by 15 μl of elution buffer (sodium vanadate, sodium tungstate, or sodium molybdate)(E) as described in Table 1.
6. Used high concentration salt to clean the resin. Added 15 μl of 2.5 M NaCl to each resin and collected the solution after centrifugation (S). At the high concentration of salt, it disrupted most of the non-covalent interaction.
7. Run the DNA electrophoresis on the flow through using pBR322 Msp I digested DNA as the molecular ladder.

TABLE 1
Elution buffer samples were prepared as follows.

| | Elution | | pH of |
|---|---|---|---|
| Sample | buffer | Formulation | elution buffer |
| 1 | 50 mM PXA1A2.1 | decavanadate | 5.7 |
| 2 | 50 mM sodium tungstate | WO4(2−) | 6.3 |
| 3 | 50 mM sodium molybdate | MoO4(2−) | 6.1 |
| 4 | 50 mM sodium molybdate | Mo7O24(6−) | 4.1 |
| 5 | 50 mM sodium molybdate | Mo8O26(4−) | 2.1 |

The gel photo (FIG. 7) and Table 2 show that pBR322 could be eluted by sodium vanadate, sodium tungstate and sodium molybdate.

TABLE 2
Summary of elution of pBR322

| | Flow | | | Able to elute |
|---|---|---|---|---|
| | through | Wash | Elution | nucleic acid |
| Vanadate pH 5.7 | 1F | 1W | 1E | Yes |
| Tungstate pH 6.3 | 2F | 2W | 2E | Yes |
| Molybdate pH 6.1 | 3F | 3W | 3E | No |
| Molybdate pH 4.1 | 4F | 4W | 4E | Yes |
| Molybdate pH 2.1 | 5F | 5W | 5E | Yes |

L: molecular ladder.

The ability of elution of molybdate depends on the pH value. At pH 6.1, the molybdate did not produce visible bands in the eluted fraction (3E).

### Example 8

Direct gPCR Reaction Using Nucleic Acid Eluted by Tungstate

Reagents: pBR322 plasmid, Bioline SensiFast Probe qPCR master mix, qPCR primer set and hydrolysis probe synthesized by IDT, Tungstate elution buffer, and vanadate elution solution.

### Steps:

1. Prepared 25 μl streptomycin conjugated resin in each spin column.
2. Removed the storage buffer and washed with 25 μl water.
3. Mixed the resin with 1000 copies of pBR322 in 0.5 M sodium chloride.
4. Wash the resin with 25 μl water
5. Elute the nucleic acid using 12.5 μl of 100 mM vanadate or 12.5 μl of 100 mM tungstate elution buffer.
6. Mixed the eluted nucleic acid with 15 μl of 2× sensiFast Probe qPCR master mix, primers, probes, and water.
7. Performed qPCR using ABI 7500Fast qPCR instrument.

TABLE 3
Results of qPCR assays

| Sample | qPCR Ct number |
|---|---|
| 1000 copies control | 31.07 |
| No template control | 37.96 |
| Vanadate elution buffer | 32.83 |
| Tungstate elution buffer | 32.85 |

The results show that nucleic acid eluted by tungstate and vanadate could be used directly in qPCR reactions without further purification.

### REFERENCES

The contents of the following are incorporated herein by reference:
Willem R. Boom, et al., Process for isolating nucleic acid (1991), U.S. Pat. No. 5,234,809.
Clare E. Bostock-Smith, Kevin J. Embrey and Mark S. Searle, Enhanced binding of a hexacyclic amidine analogue of Hoechst 33258 to the minor groove of DNA: 1H NMR and UV melting studies with “the decamer duplex d(GGTAATTACC)2”, Chem. Commun., 0: 121-122 (1997).
Martine Demeunynck, Christian Bailly, W. and David Wilson, Small Molecule DNA and RNA Binders: From Synthesis to Nucleic Acid Complexes, (2006), John Wiley & Sons, ISBN: 978-3-527-60566-8.
Prabal Giri, Maidul Hossain and Gopinatha Suresh Kumar, RNA specific molecules: Cytotoxic plant alkaloid palmatine binds strongly to poly(A) Bioorg Med. Chem. Lett. 16: 2364-2368 (2006).
Yang Liu, et al., Water-Mediated Binding of Agents that Target the DNA Minor Groove Am Chem Soc. 133(26): 10171-10183 (2011).
Florent Mouliere, et al., High Fragmentation Characterizes Tumour-Derived Circulating DNA, PloS One 6(9): e23418 (2011).
Jillian Phallen, et al., Direct Detection of Early-Stage Cancers Using Circulating Tumor DNA Science Translational Medicine 9: eaan2415 (2017).
Farial A. Tanious, et al., Sequence-dependent binding of bis-amidine carbazole dications to DNA Eur J Biochem. 268(12): 3455-64 (2001).
Alan S. Tracey, Gail R. Willsky, and Esther S. Takeuchi, “Vanadium: chemistry, Biochemistry, Pharmacology and Practical Applications” by CRC Press (2007), ISBN: 1-4200-4613-6, Page 21.
Molecular Basis of Specificity in Nucleic Acid-Drug Interactions: Proceedings of the Twenty-Third Jerusalem Symposium on Quantum Chemistry and Biochemistry Held in Jerusalem, Israel, May 14-17, (1990) Eds. B. Pullman and J. Jortner, Springer Science+Busines Media, B.V. ISBN 978-94-010-5657-1.
Hongjuan Xi, David Gray, Sunil Kumar and Dev P. Arya, Molecular recognition of single-stranded RNA: Neomycin binding to poly(A), FEBS Letters 583: 2269-2275 (2009).
Feifei Xing, Guangtao Song and Jinsong Ren Molecular recognition of nucleic acids: Coralyne binds strongly to poly(A) FEBS Lett. 579: 5035-5039 (2005).
Ram Chandra Yadav, et al., Berberine, a strong polyriboadenylic acid binding plant alkaloid: spectroscopic, viscometric, and thermodynamic study Bioorg. Med. Chem. 13: 165-174 (2005).

1. A process for the separation of a nucleic acid from a first nucleic acid-containing composition, comprising the steps of:
a) providing the first nucleic acid-containing composition in a first phase;
b) providing a nucleic acid binding molecule which is either (a) a cationic molecule which is an amine, amidine or other amine-derived cation, or (b) a minor groove nucleic acid-binding molecule in a second phase;
c) allowing contact to be maintained between the first phase and the second phase for a sufficient time for nucleic acids in the first nucleic acid-containing composition to bind to the nucleic acid-binding molecule;
d) separating the first phase from the second phase; and
e) treating the second phase to dissociate bound nucleic acids from the nucleic acid-binding molecule by contact with a dissociation composition comprising a polyoxometalate or an oxalate molecule to produce a second nucleic acid-containing composition.

2. (canceled)

3. The process of claim 21, wherein the cationic molecule is a cationic polymer selected from polyethyleneimine, poly-lysine, poly-arginine, an amidine-containing polymer, or a guanidino polymer.

4. (canceled)

5. (canceled)

6. The process of claim 1, wherein the cationic molecule is an amine selected from one or more of diethylaminoethyl (DEAE), trimethylammonium (QMA) or diethyl-(2-hydroxypropyl)aminoethyl (QAE) groups immobilised to a solid support.

7. The process of claim 1, wherein the nucleic acid binding molecule is a minor groove nucleic acid-binding molecule.

8. The process of claim 7, wherein the minor groove nucleic acid-binding molecule is a molecule carrying one more groups selected from amidino, amido, or guanidino, which binds the minor groove of DNA.

9. (canceled)

10. (canceled)

11. (canceled)

12. (canceled)

13. (canceled)

14. (canceled)

15. The process of claim 1, wherein the dissociation composition comprises a polyoxometalate ion or an oxalate.

16. The process of claim 15, wherein the dissociation composition comprises a polyoxometalate.

17. (canceled)

18. The process of claim 16, wherein one or more of the following apply:
(a) the polyoxometalate is an oxide of tungsten, molybdenum, niobium, tantalum or vanadium; and
(b) the polyoxometalate is a mixture of species formed on dissolution of the polyoxometalate in alkaline solution followed by acidification.

19. (canceled)

20. The process of claim 16, wherein the polyoxometalate vanadate is a polymer of NaVO₃.

21. (canceled)

22. The process of claim 21, wherein one or both of the following apply:
(a) the final the pH of the solution is from 5 to 6; and
(b) the concentration of the polyoxometalate in the solution is from 0.01 mM to 500 mM.

23. (canceled)

24. The process of claim 15, wherein the dissociation composition comprises an oxalate.

25. The process of claim 24, wherein one or more of the following apply:
(a) the oxalate is (NH₄)₂C₂O₄; and
(b) the concentration of oxalate is from 0.01 to 50 mM.

26. (canceled)

27. The process of claim 1, wherein one or more of the following apply:
(a) the first phase is a liquid phase; and
(b) wherein the process further comprises washing the first phase after separation from the second phase.

28. (canceled)

29. The process of claim 1, wherein the second phase is a solid phase.

30. The process of claim 29, wherein the nucleic acid binding molecule is immobilised to a solid support.

31. The process of claim 1, wherein the second phase is a liquid phase.

32. The process of claim 1, wherein one or more of the following apply:
(a) no solvent or alcohol is required to further purify the dissociated nucleic acids;
(b) the first nucleic acid-containing composition comprises materials other than nucleic acids;
(c) the first nucleic acid-containing composition is derived from a biological sample;
(d) the second nucleic acid-containing composition is substantially pure;
(e) the second nucleic acid-containing composition is substantially pure;
wherein the process further comprises collecting the second nucleic acid-containing composition;
(g) wherein the process further comprises amplifying one or more of the nucleic acids present in the second nucleic acid-containing composition; and
(h) wherein the process further comprises amplifying one or more of the nucleic acids present in the second nucleic acid-containing composition, and detecting the presence of a specific nucleic acid or of nucleic acids generally.

33. (canceled)

34. (canceled)

35. (canceled)

36. (canceled)

37. (canceled)

38. (canceled)

39. A device for the separation of a nucleic acid from a first nucleic acid-containing composition configured for use in the process of claim 1.

40. A kit comprising a first composition comprising a nucleic acid binding molecule which is either (a) a cationic molecule which is an amine, amidine or other amine-derived cation, or (b) a minor groove nucleic acid-binding molecule; and a second composition comprising a polyoxometalate or an oxalate, wherein addition of the second composition to nucleic acids bound to the nucleic acid binding molecule results in disassociation of the nucleic acids from the nucleic acid binding molecule.

41. The kit of claim 40, wherein one or more of the following apply:
(a) the nucleic acid binding molecule is immobilised on a solid support;
(b) the kit further comprises buffers and reagents for isolating a nucleic acid and/or further comprising buffers and reagents for amplification and/or detection; and
(c) the kit further comprises a device for isolating and/or amplifying and/or detecting a nucleic acid.

42. (canceled)

43. (canceled)
